(19) United States
(12) Patent Application Publication
(10) Pub. No.: US 2006/0052746 A1
(43) Pub. Date: Mar. 9, 2006

(54) Breast pump

(76) Inventor: Ji-Min Liao, Taichung (TW)

Correspondence Address: NIKOLAI & MERSEREAU, P.A., 900 SECOND AVENUE SOUTH, SUITE 820, MINNEAPOLIS, MN, 55402, US

(21) Appl. No.: 10/934,123

(22) Filed: Sep. 7, 2004

(51) Int. Cl. A61M 1/06

(52) U.S. Cl. 604/74

(57) ABSTRACT

A breast pump is disclosed to include a breast milk container, a teat cup attachable to a woman's breast, a motor, a pump driven by the motor to pump breast milk out of the teat of a woman's breast into the breast milk container through the teat cup, an electromagnetic valve installed in between the teat cup and the pump in air communication between the breast milk transferring passage and the atmosphere, and a programmable control IC chip board, which controls the electromagnetic valve to close/open the air passage between the atmosphere and the breast milk transferring passage intermittently so as to smoothen the breast milk pumping action.

### BACKGROUND OF THE INVENTION

1. Field of the Invention

The present invention relates to a breast pump for pumping breast milk out of a woman's breast and more particularly, to a programmable breast pump.

2. Description of the Related Art

A conventional breast pump, as shown in FIG. 1, comprises a breast milk container 1 and a pump unit 2. The breast milk container 1 is coupled to the output side of the transfer pump 20 of the pump unit 2 for receiving breast milk from the woman's breast. The pump unit 2 further comprises a motor 21 adapted to drive the transfer pump 20, and a teat cup 22 coupled to the input side of the transfer pump 20 and attachable to a woman's breast. During operation of the motor 21, the transfer pump 20 is reciprocated to suck in breast milk from the breast through the teat cup 22 and to transfer breast milk from the teat cup 22 to the breast milk container 1. Various breast pumps have been disclosed and have appeared on the market. These conventional breast pumps are functional. However, these conventional breast pumps are not orthopedically engineered for comfortable use. Because these conventional breast pumps repeatedly run a suction action during operation, the breast milk pumping efficiency is low and, the user's breast will feel uncomfortable.

Therefore, it is desirable to provide a breast pump that eliminates the aforesaid drawbacks.

### SUMMARY OF THE INVENTION

The present invention has been accomplished under the circumstances in view. It is the main object of the present invention to provide a breast pump, which is orthopedically engineered to provide comfort of use. It is another object of the present invention to provide a breast pump, which greatly improves breast milk pumping efficiency. To achieve these and other object of the present invention, the breast pump comprises a breast milk container, a pump unit, which comprises a teat cup attachable to a woman's breast, a motor, and a pump driven by the motor to such breast milk from the teat of the woman's breast into the breast milk container through the teat cup via a breast milk transferring passage, an electromagnetic valve installed in the breast milk transferring passage in air communication between the breast milk transferring passage and the atmosphere, and a programmable control IC chip board adapted to control the electromagnetic valve to close/open the air passage between the atmosphere and the breast milk transferring passage intermittently subject to a predetermined frequency.

### BRIEF DESCRIPTION OF THE DRAWINGS

FIG. 1 is an exploded view of a breast pump according to the prior art.

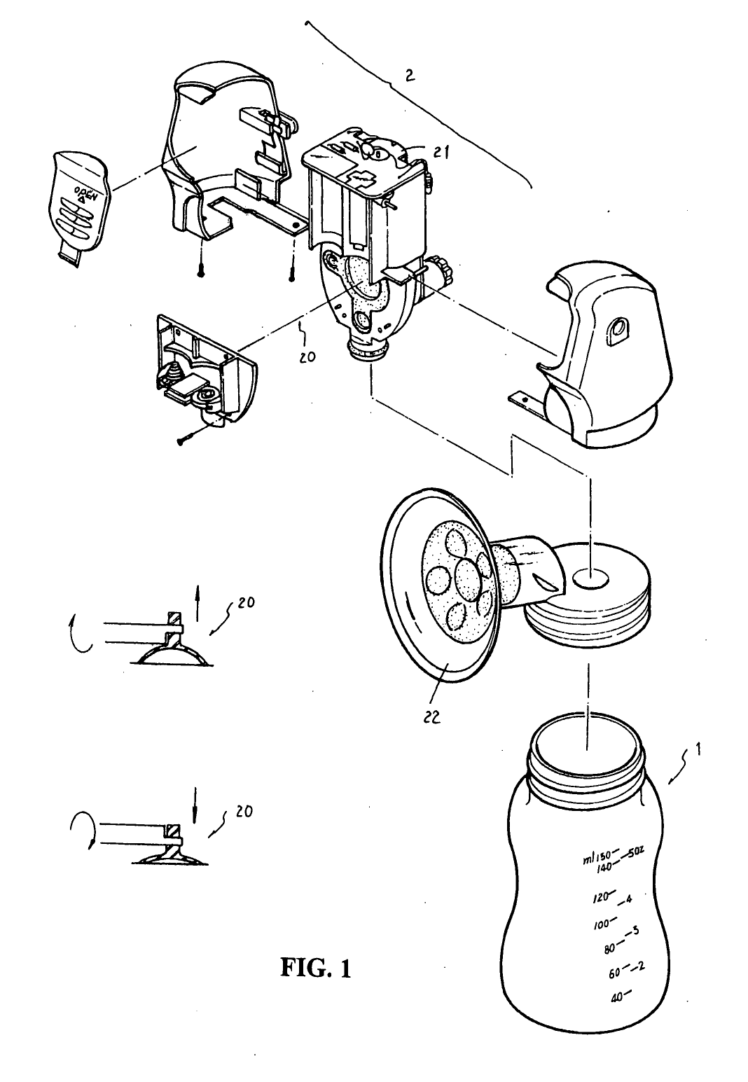

FIG. 2 is an exploded view of a breast pump according to the present invention.

FIG. 3 is an elevational assembly view of the breast pump according to the present invention.

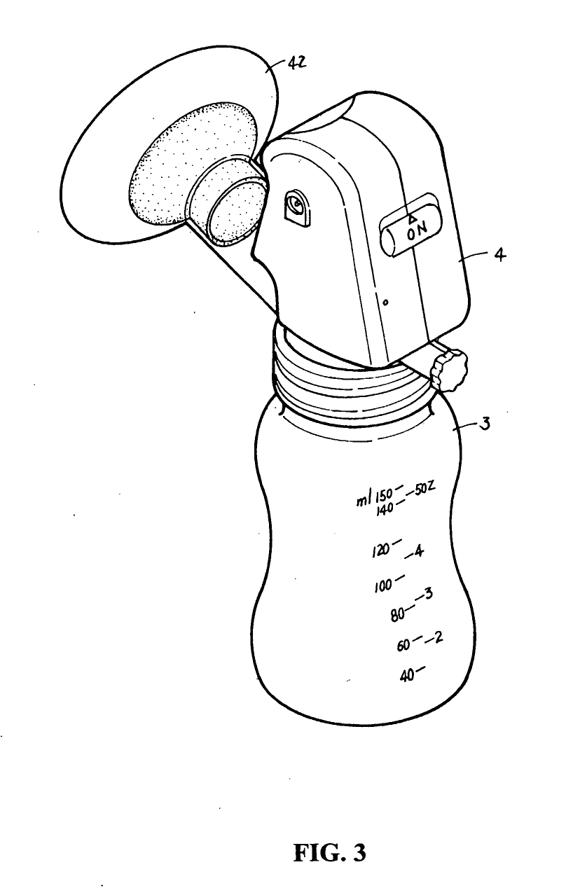

FIG. 4 is a circuit diagram of the control IC chip board according to the present invention.

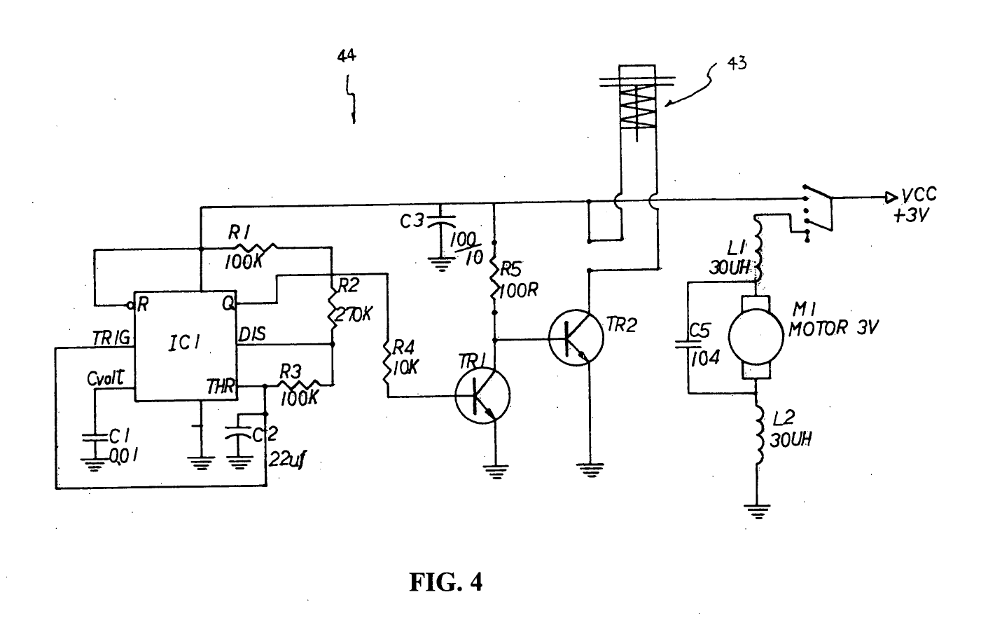

### DETAILED DESCRIPTION OF THE INVENTION

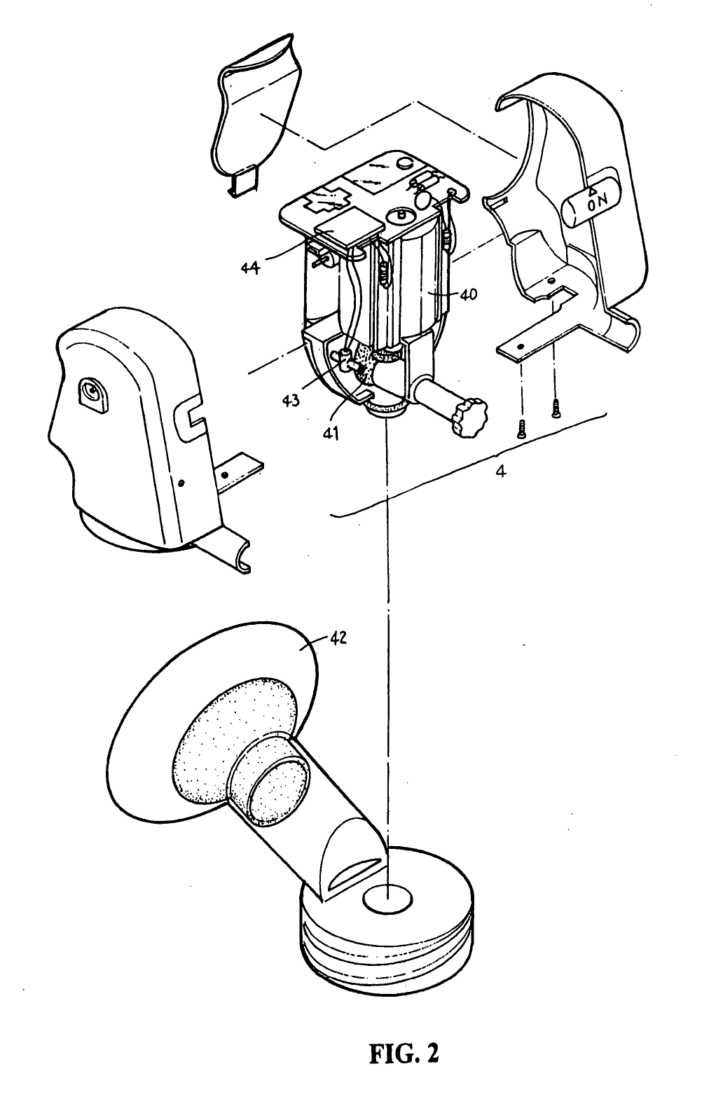

Referring to FIGS. 2˜4, a breast pump in accordance with the present invention is shown comprised of a breast milk container 3 and a pump unit 4. The pump unit 4 comprises a motor 40, and a transfer pump 41. The transfer pump 41 is a balloon type transfer pump having the suction side mounted with a teat cup 42. By means of the pumping action of the transfer pump 41, breast milk is sucked into the teat cup 42 and transferred to the breast milk container 3. Further, an electromagnetic valve (one-way check valve) 43 is installed in the breast mild transferring passage, preferably in the suction side adjacent to the transfer pump 41 for guiding in outside fresh air from the atmosphere into the breast milk transferring passage, and controlled to intermittently close/open the passage by a programmable control IC chip board 44. The optimum operation mode of the transfer pump 41 is close the passage for 0.3 second and then to open the passage for 0.7 second. During every 0.7-second open stage, fresh air is sucked into the breast milk transferring passage to release the teat suction force.

It is to be understood that the teat suction and release mode of the breast pump is not the only feature of the present invention. The main technical concept of the present invention is the use of the electromagnetic valve with the control IC chip board to provide a unique pumping action to suck in breast milk smoothly and efficiently. Without changing the basic structural design of a conventional breast pump, the invention allows the user to selectively operate the breast pump between a manual operation mode and a program-controlled operation mode. Further, because the programmable control IC chip board 44 is programmable, the user can adjust the frequency of the suction (close) and release (open) modes to fit individual requirements.

As indicated above, the breast pump is programmably controllable to produce sucking and releasing actions alternatively at a respective predetermined frequency. This orthopedically engineered design of breast pump achieves a satisfactory breast milk pumping effective and simultaneously massages the breast.

Further, a selector switch (not shown) may be installed for enabling the user to select the manual operation mode or automatic operation mode, or to select the frequencies of the suction and releasing actions.

A prototype of breast pump has been constructed with the features of FIGS. 2˜4. The breast pump functions smoothly to provide all of the features discussed earlier.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. A breast pump comprising a breast milk container and a pump unit, said pump unit comprising a teat cup attachable to a woman's breast, a motor, and a pump driven by said motor to pump breast milk out of the teat of a woman's breast into said breast milk container through said teat cup via a breast milk transferring passage, wherein the breast pump further comprises valve means installed in said breast milk transferring passage in air communication between said breast milk transferring passage and the atmosphere, and a programmable control IC chip board adapted to control said valve means to close/open the air passage between the atmosphere and said breast milk transferring passage intermittently.

2. The breast pump as claimed in claim 1, wherein said valve means is an one-way check valve, which allows air to pass from the atmosphere into said breast milk transferring passage when opened.

3. The breast pump as claimed in claim 1, said valve means is an electromagnetic valve.
